(19) United States
(12) Patent Application Publication
(10) Pub. No.: US 2019/0261595 A1
(43) Pub. Date: Aug. 29, 2019

(54) CAT-PAN LITTER-LESS LINER PRODUCT AND METHOD

(71) Applicant: Lora Langefels, New Orleans, LA (US)

(72) Inventor: Lora Langefels, New Orleans, LA (US)

(21) Appl. No.: 16/411,979

(22) Filed: May 14, 2019

Related U.S. Application Data
(63) Continuation-in-part of application No. 15/401,785, filed on Jan. 9, 2017, now abandoned.

(51) Int. Cl. A01K 1/015; A01K 1/01; B65F 1/00; A61B 10/00; G01N 31/22

(52) U.S. Cl. CPC A01K 1/0157; A01K 1/0107; B65F 1/0026; A61B 2503/40; A61B 10/007; G01N 31/22; B65F 2240/136; A61B 10/0038

(57) ABSTRACT

A monitoring disposable litter-less domestic-pet pad system and method for managing excreta of domestic pets, such as cats and dogs, performing tests for materials present in the excreta, indicating presence of such materials, and providing for secure and sanitary disposal, comprising an impermeable liner with an affixed absorbent pad having one or more incorporated indicator areas, where the indicator areas provide a level of indication ranging from a general overall level of concern in an alerting embodiment to a more detailed indication suitable for analysis by a veterinary practitioner in a diagnostic embodiment. The monitoring disposable litter-less domestic-pet pad system and method does not contaminate, denature, or otherwise compromise the urine or stool excreta, and therefore provides a secure and pleasant means of containing, saving, and transporting any such samples which are thought to warrant further analysis.

### CROSS-REFERENCE TO RELATED APPLICATIONS

This application is a continuation-in-part of pending application Ser. No. 15/401,785, filed on Jan. 9, 2017, the full disclosure of which is incorporated by reference herein and priority of which is hereby claimed.

### BACKGROUND OF THE INVENTION

This invention is a monitoring disposable litter-less domestic-pet pad system and method for managing excreta of domestic pets, such as cats and dogs, performing tests for materials present in the excreta, indicating presence of such materials, and providing for secure and sanitary disposal.

Living with domestic pets, such as cats and dogs, and also ferrets, guinea pigs, rabbits, mice and rats, and the like, involves the management of excreta—urine and feces. Cats are inclined to use a designated cat pan. Dogs do not have such an inclination. However, in circumstances where a dog is confined in a cage or pen, such as when under observation in a veterinary facility or a shelter, the dog is likely to use a specific remote corner of the area for elimination.

Analysis of urine and feces in humans and in animals can provide a wealth of health information to a qualified analyst. With humans, more understanding and cooperation in the collection of samples can be expected. But with animals, such collection can be much more difficult. The animal is unlikely to be inclined to eliminate on command, and is likely to eliminate in a location and conditions which would tend to contaminate, denature, and otherwise render the samples unfit for analysis.

The use of kitty litter in a cat pan, which is presently the most commonly used method of management, comes with several disadvantages. Packages of the litter are bulky and heavy, and have to be purchased and carried often. The litter is made of fine granules which can become airborne. When used in a cat pan, the litter tends to be scattered or spilled outside the cat pan by the cat's movements and by other factors. The cat's excreta are also scattered and spilled outside the pan. Scooping used litter for disposal stirs it up and further causes scattering or spilling of both litter and cat excreta. The resulting used litter is difficult to dispose of, as it may clog plumbing, and the process of disposal tends to further scatter or spill the litter and excreta. Even the process of pouring new litter to the cat pan can lead to scattering and spillage.

Another disadvantage of kitty litter is that it contaminates and ruins the ability to examine or analyze the cat's excreta for veterinary health purposes. Typically, a veterinarian has to catheterize the cat to retrieve a urine sample, inserting a needle into the bladder of the cat. Cat owners are often instructed to examine the urine or fecal sample of the pet to ensure that the pet remains healthy after a visit to the veterinarian. At home, regular visual checking of the cat's excreta, such as for the presence of blood or worms, is a good idea, but contamination from kitty litter interferes with the ability to do this checking.

A number of issued patents disclose the use of a multi-layered liner meant to be placed in a cat pan, with ends that may stretch over the lid of the cat pan for easy application and removal. Additionally, there are several patents that disclose the use of an odor eliminator and/or attractive scent meant to attract the cat to the pan.

For example, U.S. Pat. No. 4,869,204 was issued Sep. 26, 1989 to Patrick Yananton for an “Odorless Animal Litter Unit Having Securing Means.” The claw-resistant sorbent pad laminate is employed for the collection of animal urine. The laminate of the Yananton unit includes a bottom sheet layer of moisture-impermeable material, an intermediate sorbent layer of material having a high sorbency capacity for urine, and a top claw resistant screen means. The laminate is secured to a container by means of securing means around the periphery of the bottom sheet layer. In one embodiment of the invention, the securing means is an elasticized area or areas around the periphery of the moisture-impermeable material. In an alternate embodiment, a drawstring is movably affixed to the periphery of the bottom sheet layer.

U.S. Pat. No. 4,836,141, issued Jun. 6, 1989 to Roxana R. Whitfield for an “Odor Control Pet Excrement Pan,” covers an odor-controlling pet excrement container having a substantially flat pan. In the pan, a conforming protective liner of flexible sheet material is removably mounted, the liner having a layer of absorptive material and being adapted to contain a layer of litter material if desired. After use, the liner and its contents are removed from the pan and the excrement and litter are sealed within the liner by means of a drawstring. Super absorbent material capable of gelling liquid urine and suitable odor suppressants are incorporated in the liner and its contents.

U.S. Pat. No. 5,850,798 was issued Dec. 22, 1998 to Eric M. Engel for a “cat-pan Liner.” The invention covers a liner for a cat pan having sides extending upwardly from a bottom to a vertical height. The liner comprises a bag for placement in the cat pan. The bag comprises a bottom portion for lining the cat pan, and a contiguous top portion. A fluid-impermeable layer is placed within the box, located within the bottom portion of the bag and bonded to the bag. A sorbent layer is placed above the fluid-impermeable layer. A clay-resistant layer is placed above the sorbent layer and affixed to the fluid-impermeable layer so as to stiffen the affixed layers, whereby they tend to stay in place in the cat pan. The top portion of the bag drapes over the sides of the box for a drape length and provides ballast for helping the liner to stay in place.

U.S. Pat. No. 4,919,078, issued Apr. 24, 1990 to Glenn E. Morrison for a “Litter Packaging System,” discloses a pet litter packaging apparatus that includes a rectilinear cardboard container whose flaps expand to form a litter box having an interior of sufficient size to accommodate a pet. Litter bags lie on their sides with their openings directed against a vertical door. Beams are used to hold the bags open and to support the flaps in a house-like configuration. The pet eliminates within the bags, which can be disposed of one after the other without the pet owner having to transfer the litter.

U.S. Pat. No. 4,858,559, issued Aug. 22, 1989 to Pamela J. Allen for a “Disposable Pet Toilet Unit,” covers a disposable pet toilet unit having a body portion within which a deodorizing, absorbent, granular material is contained for use by household pets to defecate and urinate. The body portion is made of a moisture-proof material and includes a bottom and a plurality of sides extending upwardly from the bottom to form an opening. A moisture-proof film is secured to the body portion and gatherable about the opening thereof by a closure, such as a drawstring. Thus, when the deodorizing, absorbent, granular material becomes sufficiently soiled so as to no longer adequately deodorize and absorb odor and moisture, the drawstring may be pulled to gather the film together about the opening to substantially seal it. In this manner, the disposable pet toilet unit may be transported and discarded without spillage or leakage of the soiled, deodorizing, absorbent, granular material.

U.S. Pat. No. 4,813,374 was issued Mar. 21, 1989 to Claude R. Sides for a “Prepackaged Liner and Litter Assembly.” The assembly for animal waste includes a bag with a plurality of liner and litter units within the bag. Each liner and litter unit includes a liner of flexible material with the litter contained therein. Within the bag, each liner is configured in pouch configuration to facilitate holding the litter therein, and each liner is adapted to being opened at the top and arranged within a litter box to extend over the bottom and sides of the box. The liner and litter units are arranged within the bag with the tops of the liners toward the top of the bag.

U.S. Pat. No. 4,807,563 was issued Feb. 28, 1989 to Ronald L. Berry and Linda J. Scholes-Witty on a “Self-Contained, Disposable, Sanitary Toilet Facility for Pets.” The facility covers a pet litter box, including a device for locking the box after it has been used and re-closed, formed from a single foldable blank. When the box is closed or re-closed, it has inter-laying first and second halves, each having bottom and sidewall portions. The halves are connected to one another by a joining portion, and joining sidewalls. Each of the sidewalls of the first half carries a tab. The sidewall portions of the second half each define a diagonal crease, and the pair of joining side-wall portions each define a diagonal crease and a pair of vertical creases. When the box is fully open its first, second, and joining portions define an open tray. After the system has been used, the box can be re-closed and locked. During re-closing, the joining sidewall portions and the two sidewalls of the second half all fold inwardly at their creases, thereby forming a pair of slots between the pairs of creases on each side. Then, by inserting the tabs carried by the first half sidewalls into the slots which are formed between the creases, re-closing and locking of the box is completed. The box blank may include a flap for securing the box closed before it is utilized, and a handle, and may also include all items which are necessary for pet toiletry, such as a bag of litter, and a liner sized to fit over the open tray.

U.S. Pat. No. 4,756,273, issued Jul. 12, 1988 to Patrick Yananton, covers a “Reversible Bag for Use with Litter Bag.” In this second Yananton invention, a disposable animal litter pad is combined with a moisture-impermeable bag for easy disposal. The litter pad includes a sorbent pad laminate for the collection of animal urine and a top claw-resistant screen made of a urine-permeable, flexible member of material substantially inert to urine. The disposable animal litter pad laminate has a bottom layer of two sheets of moisture-impermeable material peripherally sealed together along three sides to form a bag-like structure. The sorbent pad laminate also includes a sorbent layer of material, positioned adjacent the moisture-impermeable material, and having a high sorbtion capacity for urine. Disposal is accomplished by reversing the bag over the litter pad, thereby enclosing the litter pad, along with the animal waste and litter and facilitating disposal.

U.S. Pat. No. 4,819,580 was issued on Apr. 11, 1989 to David A. Foldes for a “Disposable Cat Litter Box.” The open-top receptacle is embraced with a tube of waterproof polymer film material. The tube extends beyond the receptacle and is sufficiently long and large so that when it is desired to dispose of the litter box, the tube is inverted over the entire litter box to enclose and seal it. The disposable cat litter box comprises (1) a receptacle having a bottom and having a top edge mounted on the bottom and extending above the bottom, with the top edge defining an interior space within the receptacle, which is open at the top; and (2) a flexible waterproof tube, the tube having a portion that is sufficiently large to receive therein the receptacle, and being sufficiently large so that the tube portion can extend below the top edge of the receptacle to form a recess within and below the top edge of the receptacle to receive litter; where the tube has a closed end thereon and the receptacle being positioned within the tube portion adjacent the closed end of the tube; and where the tube extends away from the receptacle to an open end, the tube having an intermediate closure between the portion of the tube which contains the receptacle and the open end, the tube being sufficiently long from the intermediate closure so that the open end of the tube can be inverted over the portion of the tube that contains the receptacle to enclose the receptacle and the recess in the receptacle formed by the tube.

U.S. Pat. No. 4,840,140 was issued on Jun. 20, 1989 to Patrick Yananton and Janice P. Parker for a “Securing Means for [an] Odorless Animal Litter Unit.” The invention covers a combination of a litter device for use with cats, which has a sorbent pad laminate for the collection of animal urine. The device consists of a bottom sheet layer of moisture-impermeable material in contact with the base and walls of the litter device, an intermediate sorbent layer of material having a high absorbency capacity for urine, a top claw-resistant screen, and securing device. The securing device removably affixes the sorbent pad laminate to the litter device, thereby preventing the relative movement between the sorbent pad and the litter device that can be caused by the clawing action of an animal.

U.S. Pat. No. 5,526,770, issued on Jun. 18, 1996 to Theodore M. Kiebke for a “Biodegradable Dustless Cat Litter,” covers a dustless cat litter that includes a wetting additive to agglomerate dust to litter ingredients and promote hydration. Exemplary wetting additives are water soluble, anionic, amphoteric or synthetic surfactants, surfactant mixtures, soaps, or synthetic detergents, which are applied to the litter ingredients to bind dust and fines contained in the mixtures. A masking fragrance, neutralizer, or attractant may be admixed with the surfactant. Exemplary hydrating cat litter or additive mixtures, which solidify upon contact with liquids to encapsulate pet waste matter, include a granulated base material (e.g., whole or coarse-ground agricultural grains, pulse crops, clay granules, or mixtures thereof), a gluten-containing material (e.g., semolina or ground wheat), and may include sodium bicarbonate and/or a carrier bound fragrance. A quantity of surfactant on the order of ½ to 7 ounces per 10 pounds litter containing 5% to 10% fines is applied to the ingredients during blending of the mixture.

### SUMMARY OF THE INVENTION

This invention provides a monitoring disposable litter-less domestic-pet pad system and method for managing excreta of domestic pets, such as cats and dogs, performing tests for materials present in the excreta, indicating presence of such materials, and providing for secure and sanitary disposal, comprising an impermeable liner with an affixed absorbent pad having one or more incorporated indicator areas, where the indicator areas provide a level of indication ranging from a general overall level of concern in an alerting embodiment to a more detailed indication suitable for analysis by a veterinary practitioner in a diagnostic embodiment. The monitoring disposable litter-less domestic-pet pad system and method does not contaminate, denature, or otherwise compromise the urine or stool excreta, and therefore provides a secure and pleasant means of containing, saving, and transporting any such samples which are thought to warrant further analysis.

### BRIEF DESCRIPTION OF THE DRAWINGS

Reference will now be made to the drawings, wherein like parts are designated by like numerals, and wherein:

FIG. 1 is a perspective schematic view of a first embodiment of the monitoring disposable litter-less domestic-pet pad of the invention;

FIG. 2 is an exploded schematic view of a first embodiment of the monitoring disposable litter-less domestic-pet pad of the invention;

FIG. 3 is a perspective schematic view of a second embodiment of the monitoring disposable litter-less domestic-pet pad of the invention;

FIG. 4 is an exploded schematic view of a second embodiment of the monitoring disposable litter-less domestic-pet pad of the invention;

FIG. 5 is a perspective schematic view of an alerting embodiment of the monitoring disposable litter-less domestic-pet pad of the invention, in use, not showing any alert;

FIG. 6 is a perspective schematic view of an alerting embodiment of the monitoring disposable litter-less domestic-pet pad of the invention, in use, showing an alert;

FIG. 7 is a perspective schematic view of a diagnostic embodiment of the monitoring disposable litter-less domestic-pet pad of the invention, in use, not showing any results; and

FIG. 8 is a perspective schematic view of a diagnostic embodiment of the monitoring disposable litter-less domestic-pet pad of the invention, in use, showing results.

### DETAILED DESCRIPTION OF THE INVENTION

The monitoring disposable litter-less domestic-pet pad 10 provides an impermeable liner 1 onto which is fixed an absorbent pad 2 which incorporates at least one indicator area 3. The domestic pet eliminates onto the absorbent pad 2. The impermeable liner 1 prevents the excreta from passing through and soiling the underlying surface, and later will serve as a bag or envelope for either disposing of the waste or preserving it for further analysis, if warranted. The indicator area 3 or areas provide a visual indication of, variously, the presence, absence, or concentrations of one or more components detected in the excreta. Typical components of significance are known in the veterinary and medical arts, and are such as acids or bases, sugars, proteins, ketones, nitrites, blood, bilirubin, leukocytes, and others. In a more simple, alerting embodiment, the presence, absence, or abnormal concentration of a defined component or combination of components will cause a visual mark or symbol to appear—in the nature of a litmus-test reaction. The visual mark could be some sort of symbol which would be understood to indicate whether or not further investigation into the health of the animal is warranted. The visual mark could be words of instruction. This alerting embodiment would be appropriate for lay persons, and would, ideally, suggest further investigation without causing undue alarm. In a more complex, diagnostic embodiment, the indicator area 3 is divided into more granular specific indicators 5 of the components and concentrations contained in the excreta. Such a diagnostic embodiment is appropriate for, as examples, a veterinary facility or a shelter having regular access to qualified diagnosticians and analysts.

When the monitoring disposable litter-less domestic-pet pad 10 is used with cats, it will likely be used in a cat pan, and it will negate the need for kitty litter, and therefore the disadvantages of kitty litter. When used with dogs, it will likely be used under conditions where the dog is confined—such as for examination—and the dog is more likely to use such a pad placed in a remote corner. The illustrations and examples are given with regard to cats but are applicable to dogs and other domestic-pet animals.

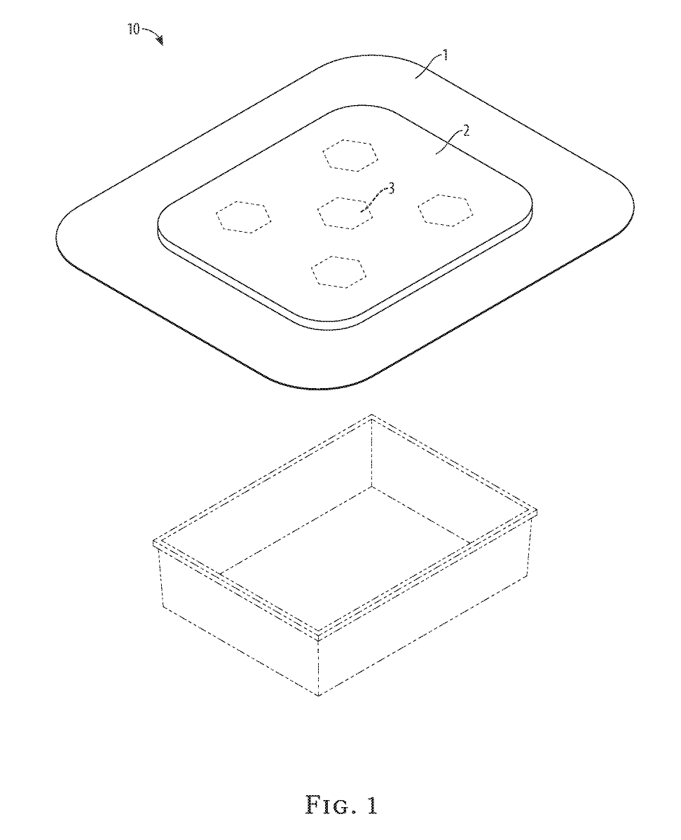

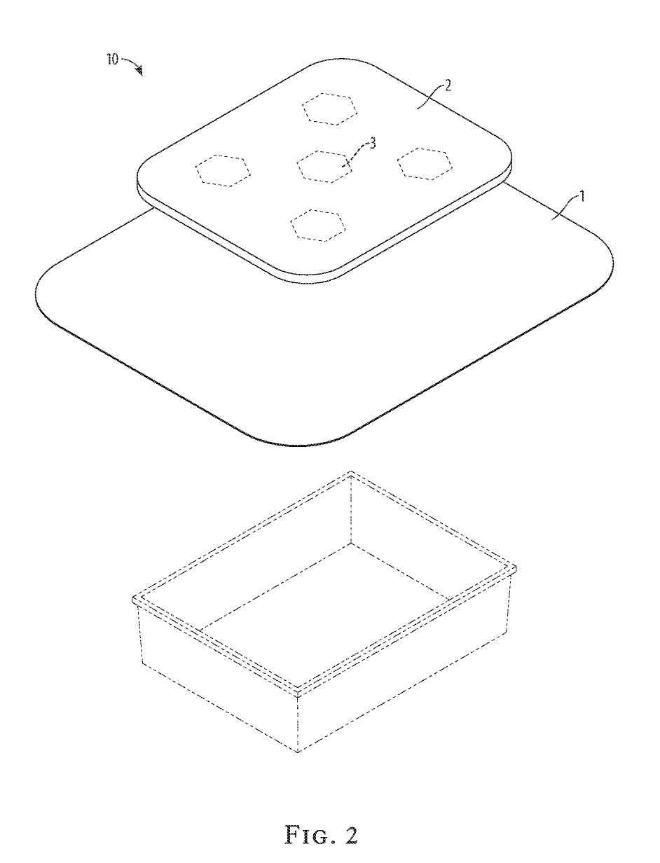

Referring to FIG. 1 and FIG. 2, showing a first embodiment of the monitoring disposable litter-less domestic-pet pad 10 of the invention, the monitoring disposable litter-less domestic-pet pad 10 is meant to be placed into a standard cat pan, which is generally between one and two feet in each lateral dimension, covering an area of approximately two to three square feet, and a depth of from two to five inches. The monitoring disposable litter-less domestic-pet pad 10 completely replaces and eliminates the use of kitty litter, which eliminates the purchase and handling of litter, the dust of litter, the scattering of litter and waste, and the contamination of cat excreta making it useless for health analysis.

The monitoring disposable litter-less domestic-pet pad 10 provides an impermeable liner 1 which fits into and over the sides of a cat pan. The impermeable liner 1 contains the cat waste, and forms a disposable bag after use. The impermeable liner 1 can have an elastic edge for gripping the rim of the cat pan. The impermeable liner 1 can be made from a sheet of rubber or plastic or of rubberized or plasticized paper or textile material.

Affixed to the center of the upward face or inner face of the impermeable liner 1 is an absorbent pad 2. The absorbent pad 2 absorbs cat excreta, which keeps the excreta from scattering. The absorbent pad 2 does not contaminate nor denature the cat excreta, which can be easily and conveniently be given a spot analysis when removing the cat-pan litter-less liner 10 after the cat has used it. The absorbent pad 2 is meant to be disposed of after use, contained within the disposable bag formed by the impermeable liner 1.

The absorbent pad 2 incorporates at least one indicator area 3, which provides the indications appropriate to either the alerting embodiment or diagnostic embodiment as treated above.

In use, the monitoring disposable litter-less domestic-pet pad 10 is placed to line the bottom and sides of the cat pan, providing the cat with a place to urinate and defecate. The monitoring disposable litter-less domestic-pet pad 10 prevents spilling of any excreta or other substance from the cat pan. Periodically, the used monitoring disposable litter-less domestic-pet pad 10 is removed and either disposed of or kept for analysis. The monitoring disposable litter-less domestic-pet pad 10 can be removed and disposed of without spilling any excreta or other substance from the cat pan. During the removal and disposal process the cat excreta can be visually spot-checked for blood, parasites, or other indications of poor health.

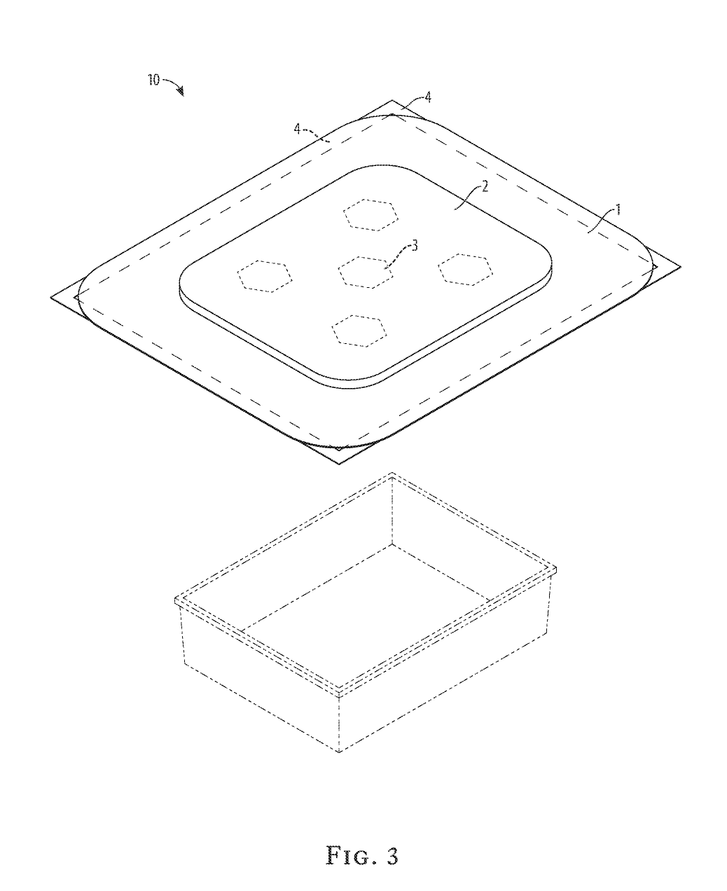

Referring to FIG. 3 and FIG. 4, showing a second embodiment of the monitoring disposable litter-less domestic-pet pad 10 of the invention, the monitoring disposable litter-less domestic-pet pad 10 can be provided with a strap or band 4 around the perimeter of the impermeable liner 1. Such a band 4 can be used as a tie strap when a used monitoring disposable litter-less domestic-pet pad 10 is being disposed of. It can also be used to gather the perimeter of the impermeable liner 1 about the rim of the cat pan. The band 4 can be made of a similar material to the impermeable liner 1. The band 4 can also be made of an elastic material that will gather the monitoring disposable litter-less domestic-pet pad 10 about the rim of the cat pan.

The impermeable liner 1 or the absorbent pad 2 of the monitoring disposable litter-less domestic-pet pad 10 can have an applied or incorporated substance that is attractive to cats. The absorbent pad 2 of the monitoring disposable litter-less domestic-pet pad 10 can have an applied or incorporated substance that neutralizes odors without denaturing or contaminating the pet excreta.

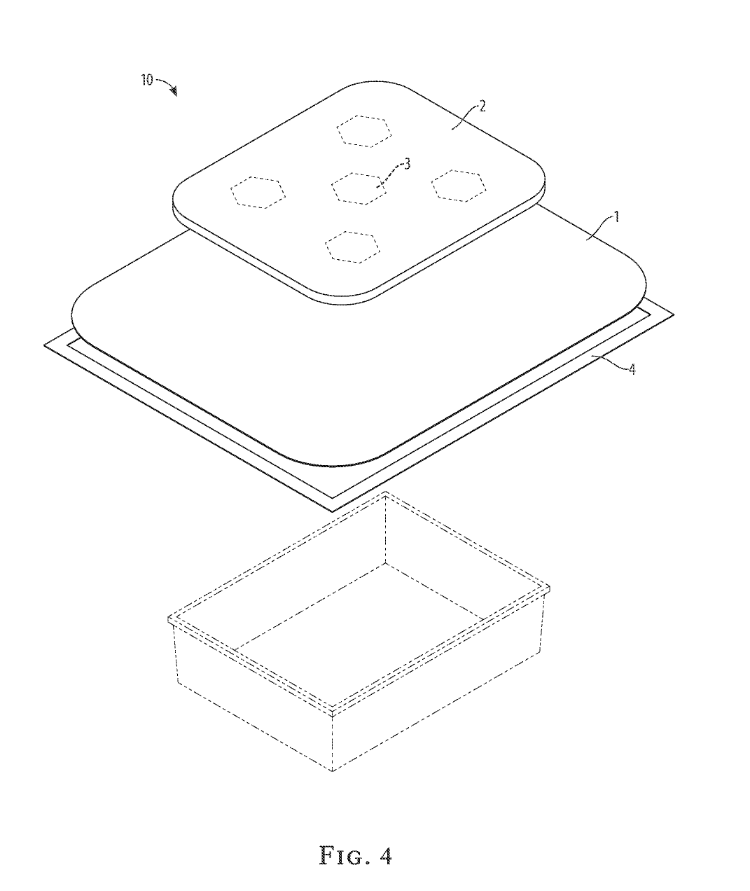

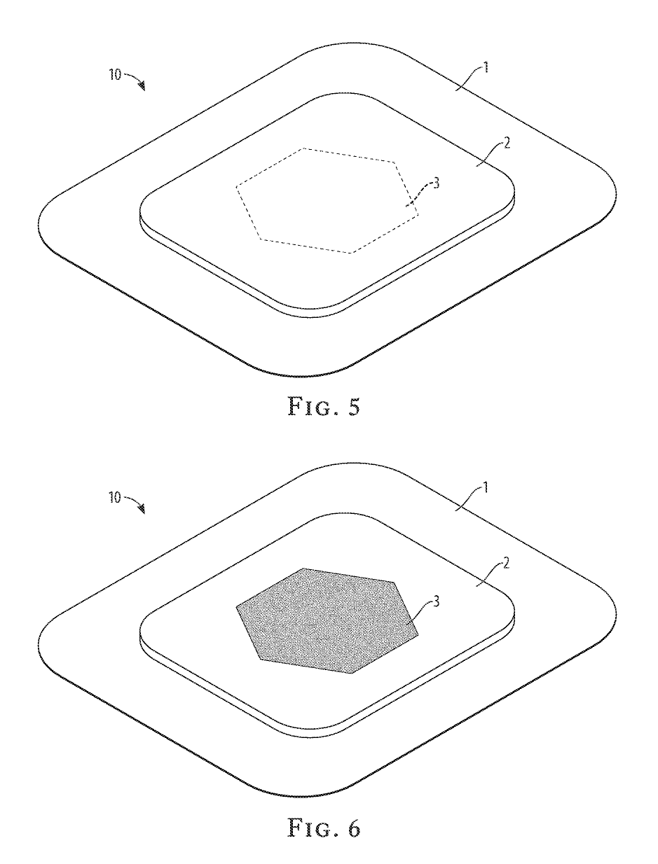

Referring to FIG. 5 and FIG. 6, in an alerting embodiment the absorbent pad 2 of the monitoring disposable litter-less domestic-pet pad 10 incorporates at least one indicator area 3, and the indicator area 3 provides a simple indication with only two, or only a few states. An example of a two-state indicator could be an area which is colorless when the component or components of interest are either not present or are present only in normal or expected concentrations, and which becomes colored when the presence or concentration deviates from normal. A three-state or four-state indicator could change to different colors under different circumstances, providing a little more information. In use, the alerting embodiment provides a way to make a daily or frequent spot check of an animal's health, both by visual inspection of the excreta and by observation of the indicator area 3 which will indicate suspected abnormality or imbalance of otherwise hard to see components. If any abnormality is suspected, the excreta, which has not been contaminated or denatured, can be sealed within the impermeable liner and given to a veterinarian for further diagnostic testing.

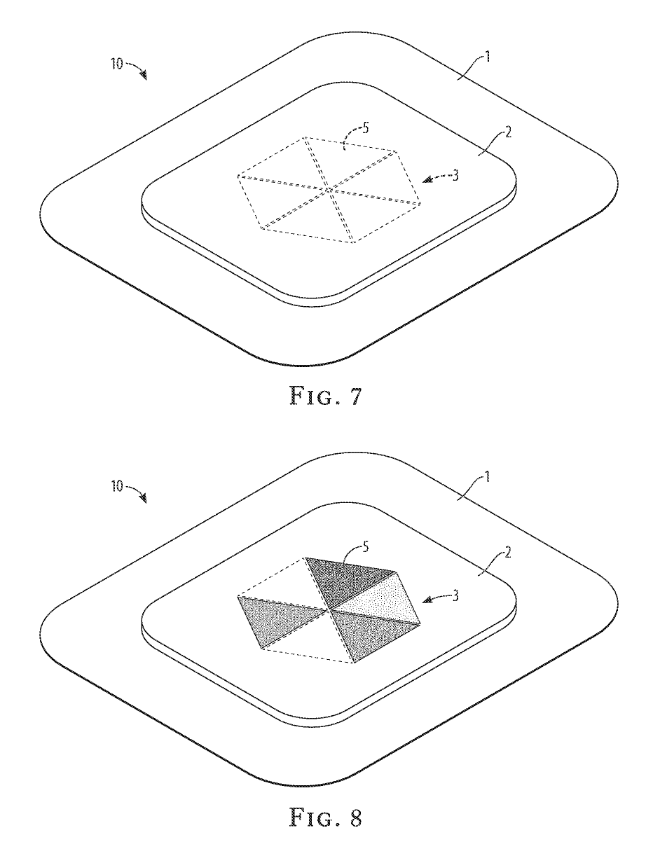

Referring to FIG. 7 and FIG. 8, in a diagnostic embodiment the absorbent pad 2 of the monitoring disposable litter-less domestic-pet pad 10 also incorporates at least one indicator area 3, and the indicator area 3 in turn provides more than one diagnostic segments 5, each of which can indicate the presence, absence, or concentration of a specific component of interest. This diagnostic embodiment will be useful in circumstances where a qualified diagnostician is available to interpret the information.

The absorbent pad 2 of the cat-pan litter-less liner 10 can have an applied or incorporated substance that reacts with cat excreta in such a way as to provide a visual indication of the presence of blood or other marker of poor health.

Many changes and modifications can be made in the present invention without departing from the spirit thereof. I therefore pray that my rights to the present invention be limited only by the scope of the appended claims.

I claim:

1. A monitoring disposable litter-less domestic-pet pad system for managing excreta of a domestic pet having a defined area for elimination, and for monitoring health as indicated by presence, absence, or concentration of components of interest, the monitoring disposable litter-less domestic-pet pad system comprising:
(i) an impermeable liner adapted to cover the defined area for elimination in use, and further adapted to form a disposable bag after use, having a perimeter, an inner face, and an outer face;
(ii) an absorbent pad affixed to said impermeable liner on the inner face, adapted to absorb pet excreta in use, and further adapted to be disposed of after use; and
(iii) at least one indicator area incorporated into said absorbent pad, adapted to react to and indicate presence, absence, or concentration of components of interest;
where, in use, said impermeable liner with said absorbent pad is placed to line the defined area of elimination, providing the pet with a place to urinate and defecate;
where, in use, said impermeable liner with said absorbent pad prevents spilling of any excreta or other substance;
where, in use, said indicator area provides a visual indication of abnormal levels of components of interest; and
where, in use, the disposable bag formed from said impermeable liner with said absorbent pad is removed and disposed of without spilling any excreta or other substance.

2. The monitoring disposable litter-less domestic-pet pad system of claim 1, further comprising a band around the perimeter of said impermeable liner.

3. The monitoring disposable litter-less domestic-pet pad system of claim 1, further adapted to allow recovery of pet excreta without degrading its utility for health analysis.

4. The monitoring disposable litter-less domestic-pet pad system of claim 1, further comprising a cat attractant incorporated into said impermeable liner.

5. The monitoring disposable litter-less domestic-pet pad system of claim 1, further comprising a cat attractant incorporated into said absorbent pad.

6. The monitoring disposable litter-less domestic-pet pad system of claim 1, further comprising an odor-eliminating substance incorporated into said absorbent pad.

7. The monitoring disposable litter-less domestic-pet pad system of claim 1, further comprising a substance that reacts with pet excreta in such a way as to provide a visual indication of the presence of blood or other marker of poor health, incorporated into said absorbent pad.

8. The monitoring disposable litter-less domestic-pet pad system of claim 1, further comprising a band around the perimeter of said impermeable liner adapted to be used as a tie strap for disposal.

9. The monitoring disposable litter-less domestic-pet pad system of claim 1, further comprising a band around the perimeter of said impermeable liner adapted to be used for gathering of the perimeter of said impermeable liner about a rim of a cat pan.

10. The monitoring disposable litter-less domestic-pet pad system of claim 1, further adapted for collection of specimens of pet waste for veterinary analysis.

11. A monitoring disposable litter-less domestic-pet pad method comprising:
(i) providing a monitoring disposable litter-less domestic-pet pad, comprising:
(a) an impermeable liner adapted to cover the defined area for elimination in use, and further adapted to form a disposable bag after use, having a perimeter, an inner face, and an outer face;
(b) an absorbent pad affixed to said impermeable liner on the inner face, adapted to absorb pet excreta in use, and further adapted to be disposed of after use; and
(c) at least one indicator area incorporated into said absorbent pad, adapted to react to and indicate presence, absence, or concentration of components of interest;
(ii) placing said monitoring disposable litter-less domestic-pet pad;
(iii) providing the pet with a place to urinate and defecate;
(iv) preventing spilling of any excreta or other substance;
(v) removing said monitoring disposable litter-less domestic-pet pad without spilling any excreta or other substance; and
(vi) disposing said monitoring disposable litter-less domestic-pet pad as the disposable bag without spilling any excreta or other substance.

12. The monitoring disposable litter-less domestic-pet pad method of claim 11, where said monitoring disposable litter-less domestic-pet pad is further comprising a band around the perimeter of said impermeable liner.

13. The monitoring disposable litter-less domestic-pet pad method of claim 11, further allowing recovery of pet excreta without degrading its utility for health analysis.

14. The monitoring disposable litter-less domestic-pet pad method of claim 11, where said monitoring disposable litter-less domestic-pet pad is further comprising a cat attractant incorporated into said impermeable liner.

15. The monitoring disposable litter-less domestic-pet pad method of claim 11, where said monitoring disposable litter-less domestic-pet pad is further comprising a cat attractant incorporated into said absorbent pad.

16. The monitoring disposable litter-less domestic-pet pad method of claim 11, where said monitoring disposable litter-less domestic-pet pad is further comprising an odor-eliminating substance incorporated into said absorbent pad.

17. The monitoring disposable litter-less domestic-pet pad method of claim 11, where said monitoring disposable litter-less domestic-pet pad is further comprising a substance that reacts with pet excreta in such a way as to provide a visual indication of the presence of blood or other marker of poor health, incorporated into said absorbent pad.

18. The monitoring disposable litter-less domestic-pet pad method of claim 11, where said monitoring disposable litter-less domestic-pet pad is further comprising a band around the perimeter of said impermeable liner adapted to be used as a tie strap for disposal.

19. The monitoring disposable litter-less domestic-pet pad method of claim 11, where said monitoring disposable litter-less domestic-pet pad is further comprising a band around the perimeter of said impermeable liner adapted to be used for gathering of the perimeter of said impermeable liner about a rim of a cat pan.

20. The monitoring disposable litter-less domestic-pet pad method of claim 11, where said monitoring disposable litter-less domestic-pet pad is further adapted for collection of specimens of pet waste for veterinary analysis.
